(19) United States
(12) Patent Application Publication
(10) Pub. No.: US 2026/0306037 A1
(43) Pub. Date: Oct. 8, 2026

(54) DETECTION SYSTEM OF IONIZING RADIATION EMITTED BY SOURCES, SUCH AS RADIOACTIVE SAMPLES AND THE LIKE, DETECTION SENSORS AND DETECTION METHOD

(71) Applicants: Alma Mater Studiorum - Università di Bologna, Bologna (IT); Azienda U.S.L. - IRCCS di Reggio Emilia, Reggio nell'Emilia (IT)

(72) Inventors: Beatrice FRABONI, Bologna (IT); Laura BASIRICÒ, Bologna (IT); Andrea CIAVATTI, Bologna (IT); Mauro IORI, Reggio nell'Emilia (IT); Federica FIORONI, Reggio nell'Emilia (IT); Lorenzo PIERGALLINI, Reggio nell'Emilia (IT)

(73) Assignees: Alma Mater Studiorum - Università di Bologna, Bologna (IT); Azienda U.S.L. - IRCCS di Reggio Emilia, Reggio nell'Emilia (IT)

(21) Appl. No.: 18/998,621

(22) PCT Filed: Jul. 19, 2023
(86) PCT No.: PCT/IT2023/050166

§ 371 Date: Jan. 27, 2025

(30) Foreign Application Priority Data
Jul. 26, 2022 (IT) 102022000015666

(51) Int. Cl. A61B 6/42; G01T 1/02; G01T 1/169

(52) U.S. Cl. CPC A61B 6/4208; G01T 1/026; G01T 1/169

(57) ABSTRACT

The present invention relates to a detection system (1, 1′, 1″) of radiation emitted by a source (C) comprising: at least one detection unit (2, 2′, 2″), having a support substrate (21), a detection layer (22), arranged on said support substrate (21) and comprising one or more detection sensors (4), to detect the radiation emitted by a contaminating agent (C) by generating an electrical signal following the interaction with the ionizing radiation, and an insulating film (23), arranged on said detection layer (22), with which the contaminating agent (C) can come into contact, so that the ionizing radiation emitted by said agent is detected by one or more detection sensors (4); and at least one interaction terminal (3), connected to said detection unit (2, 2′, 2″) and configured to detect and communicate the position of said contaminant (C) based on the detection sensors affected by the ionizing radiation emitted by said contaminant (C).

The present invention also relates to a detection unit (2, 2′, 2″) and a method for detecting radiations emitted by a source (C).

The present invention relates to a detection system of ionizing radiation emitted by sources, such as radiopharmaceuticals, radioactive samples and the like, detection sensors and detection method.

### FIELD OF INVENTION

More specifically, the invention relates to a system for detecting radioactive sources, such as radioactive samples, radiopharmaceuticals and the like, designed and manufactured in particular to detect the spatial position of radioactive sources even in small areas, such as the human body or on surfaces, which allows high spatial accuracy.

In the following, the description will be aimed, in particular, at determining the presence and position of radiopharmaceuticals or samples of radioactive substances in general, also infused inside a body, but it is clear that the same should not be considered limited to this specific use. A further example of use could be a real-time control system for centering radiation beams or personal dosimeters, for example, worn by patients during radiotherapy sessions and diagnostic examinations, or for beam centering during inspection by radiation on large surfaces of cargo and containers.

### PRIOR ART

As is well known, in work environments involving the use of unsealed radioactive substances, there are increasingly frequent risks of contamination of objects and/or surfaces.

To detect these substances, devices such as contaminometers are known in the field, suitable for detecting ionizing radiations emitted by substances of various types, including radiopharmaceuticals, diluents, and the like.

In general, these known systems do not allow real-time detection of radioactive contamination, which does not allow any damage to be promptly prevented, above all considering the medical field in the case of infusions of radioactive substances for the treatment of diseases, dosimetry during treatments with beam radiotherapy or diagnostics.

Currently, the detection of radioactive contamination is generally localized only through punctual instrumentation, i.e. at specific points, and is not extendable over a large area. Among other things, by means of the systems according to the prior art, it is not always possible to accurately detect all the contaminated points.

Thus, the detection of contamination is only possible for small areas. However, for safety reasons, a long and invasive cleaning procedure of larger areas is required, as well as the entire working environment. This aspect is particularly relevant in very large work surfaces (e.g., hot chambers, nuclear plants, and the like).

In general, the detectors currently available on the market for the detection of radioactive contamination on work surfaces are based on devices that, although portable, are bulky and allow punctual detection only upon request of the operator (proportional counters, Geiger-Muller counters, scintillators).

The perception and location of possible contamination are therefore almost completely brought to the attention of the personnel in charge. In fact, proportional Geiger counters, which are currently the main instruments for detecting diffuse contamination, as well as not being particularly handy for less easily inspected environments, cannot be extended over a large area and require high supply voltages.

The detection of radioactive contamination and its accurate cleaning is therefore highly dependent on the care and experience of the operators involved. Think, in particular, of the healthcare sector, with the consequent related safety risks in the event of human errors.

It is apparent that the aforementioned limitations are very onerous in terms of risk and, consequently, in terms of costs.

### Scope of the Invention

In the light of the above, it is, therefore, the scope of the present invention to propose a unit for detecting ionizing radiations emitted by sources, such as radiopharmaceuticals and the like, which allows overcoming the limits of the prior art, allowing locating the source of scattered radiation or beam position with high accuracy in different environments or districts.

The scope of the present invention is to allow the detection of the presence and position of the radioactive substance or of the radiation beam in real-time.

Another scope of the invention is to allow visualization of the position of the surface contaminated by the radioactive substance.

A further object of the present invention is to provide the instruments necessary for the detection of radioactive substances and the apparatuses which allow it.

### Object of the Invention

It, therefore, forms a specific object of the present invention a detection system of radiation emitted by a source comprising: at least one detection unit, having a support substrate, a detection layer, arranged on said support substrate and comprising one or more detection sensors, to detect the radiation emitted by a contaminating agent by generating an electrical signal following the interaction with the ionizing radiation, and an insulating film, arranged on said detection layer, with which the contaminating agent can come into contact, so that the ionizing radiation emitted by said agent is detected by one or more detection sensors; and at least one interaction terminal, connected to said detection unit and configured to detect and communicate the position of said contaminant based on the detection sensors affected by the ionizing radiation emitted by said contaminant.

Always according to the invention, each of said detection sensors may comprise a semiconductor material arranged on said support substrate, and at least two electrodes, obtained by metallization, connected to the semiconductor material and to said interaction terminal.

Still according to the invention, said detection sensors may be configured to have a differentiated response according to the energy spectrum emitted by said contaminant to drive said interaction terminal so as to distinguish the type of contaminant.

Advantageously according to the invention, said semiconductor material may be selected from one or more of the following: 4-hydroxycyanobenzene (4HCB); 1,8-naphthaleneimide (NTI); 1,5-dinitronaphthalene (DNN); 6,13-bis (triisopropylsilylethynyl) pentacene (TIPS-pentacene); bis (triisopropylgermylethynyl) pentacene (TIPGe-pentacene); 2,8-difluoro-5,11-bis (triethylsilylethynyl) anthradithiophene (diF-TES-ADT); 5,11-bis (triethylgermylethynyl) anthradithiophene (diF-TEGADT); polymer poly (9,9-dioctyfluorene) blended with Bismuth (III) oxide (Bi2O3) nanoparticles (NPs); Dinaphtho [2,3-b: 2′, 3′-f] thieno [3,2-b] thiophene (DNTT) and organic n-type semiconductors.

Further according to the invention, said semiconductor material may be of the perovskite hybrid type and is selected from one of the following: having a generic ABX formulation₃, wherein A=Methylammonium (MA=CH₃NH₃⁺), Formamidinium (FA=CH (NH₂)₂⁺), Cs⁺; B=Pb²⁺, Sn²⁺; X=I⁻, Br⁻, Cl⁻, in particular MAPbl₃, MAPbBr₃, mixed: Cs_{0.05}FA_{1-y}MA_{y}Pb (I₁₋ₓ Brₓ)₃); Hybrid perovskites having generic formula (R-NH₃)₂PbX₄, wherein R belongs to the alkyl or aryl group, such as PEA₂PbBr₄ and PEA₂Pbl₄, with PEA=C₆H₅C₂H₄NH₃⁺; Inorganic perovskites, such as CsPbX3 (with X=Cl, Br or I).

Preferably according to the invention, said support substrate may be plastic, flexible, insulating and waterproof, and said insulating film may be a waterproof insulating polymer, such as mylar, parylene, silicone polymers.

Always according to the invention, said detection unit may be shaped as a rectangular or square module, intended to be arranged on a flat or vertical surface.

Still according to the invention, said detection unit may have the shape of a bracelet or a band, so that it is wearable on a limb of a user, for the detection of radiation emitted by radiopharmaceuticals for the dose monitoring or the identification of radiopharmaceutical leaks.

Advantageously according to the invention, said detection unit may have the shape of plaster or patch, so that it can be applied on the surface of a user's body, for the detection of radiopharmaceutical leaks.

Further according to the invention, said detection unit may have the shape of plaster or patch, so that it can be applied on the surface of a user's body, for the centering or the dosimetry of therapeutic and diagnostic beams.

Preferably according to the invention, said interaction terminal comprises a plurality of LEDs, forming a screen, configured for activating following the detection of radiation by one or more detection devices.

Always according to the invention, each detection device may be operatively connected to a respective LED, wherein the arrangement of the LEDs on the screen corresponds to the arrangement of the corresponding detection sensors of the detection unit.

Still according to the invention, said detection system may comprise a plurality of detection units, each operatively connected to a respective LED, wherein the arrangement of the LEDs on the screen corresponds to the arrangement of the corresponding detection units.

Further according to the invention, said detection unit may comprise transceiver means for transmitting signals detected by said detection sensors, and said interaction terminal may comprise a mobile phone, tablet, and the like, equipped with a display, capable of receiving the signals transmitted by said transceiver means of the detection unit, so as to show on the display the readings of the detection units.

It is further object of the present invention a detection unit for detecting a radioactive source, comprising a support substrate, a detection layer, arranged on said support substrate, and comprising one or more detection sensors, to detect the radiation emitted by a contaminating agent by generating an electrical signal following exposure to the radiation, and an insulating film, arranged on said detection layer, with which the contaminating agent can come into contact, so that the ionizing radiation emitted by said agent is detected by one or more detection sensors.

Always according to the invention, said detection sensors may comprise a semiconductor material arranged on said support substrate, and at least two electrodes, obtained by metallization, connected to the semiconductor material and to said interaction terminal.

Still according to the invention, said semiconductor material may be selected from one or more of the following: 4-hydroxycyanobenzene (4HCB); 1,8-naphthaleneimide (NTI); 1,5-dinitronaphthalene (DNN); 6,13-bis (triisopropylsilylethynyl) pentacene (TIPS-pentacene); bis (triisopropylgermylethynyl) pentacene (TIPGe-pentacene); 2,8-difluoro-5,11-bis (triethylsilylethynyl) anthradithiophene (diF-TES-ADT); 5,11-bis (triethylgermylethynyl) anthradithiophene (diF-TEGADT); polymer poly (9,9-dioctyfluorene) blended with Bismuth (III) oxide (Bi203) nanoparticles; (NPs); Dinaphtho [2,3-b: 2′, 3′-f] thieno [3,2-b] thiophene (DNTT) and organic n-type semiconductors.

Advantageously according to the invention, said semiconductor material is of the perovskite hybrid type and is selected from one of the following: having a formulation generic ABX₃, wherein A=Methylammonium (MA=CH₂NH₃⁺), Formamidinium (FA=CH (NH₂)₂⁺), Cs⁺; B=Pb²⁺, Sn²⁺; X=I⁻, Br⁻, Cl⁻, in particular MAPbl3, MAPbBr3, mixed: CS_{0.05}FA_{1-y}MA_{y}Pb (I₁₋ₓ Brₓ)₃); Hybrid perovskites having generic formula (R-NH₃)₂PbX₄, wherein R belongs to the alkyl or aryl group, such as PEA₂PbBr₄ and PEA₂Pbl₄, with PEA=C₆H₅C₂H₄NH₃⁺; inorganic perovskites for example CsPbX₃ (with X=Cl, Br or I).

It is also object of the present invenzion a method for detecting radiations emitted from a source comprising by means of a detection system according to any one of claims 1-14, characterized in that it comprises the following steps: irradiating said insulating film with said source; detecting the radiation of said radiation source by means of one or more detection sensors of said detection layer; detecting and communicating the position of said contaminating agent by means of said interaction terminal, based on the detection sensors affected by the ionizing radiation emitted by said agent.

### BRIEF DESCRIPTION OF THE FIGURES

The present invention will be now described, for illustrative but not limitative purposes, according to its preferred embodiments, with particular reference to the figures of the enclosed drawings, wherein:

FIG. 1 shows a diagram of a unit for detecting radiation emitted by sources according to the present invention;

FIG. 2 shows a diagram of a semiconductor detection device of the detection unit of FIG. 1;

FIG. 3A shows the graph of the current intensity variation in response of an organic/hybrid semiconductor to the radiation emitted by the radioisotope Fluorine-18;

FIG. 3B shows the chart of the current intensity variation in response of an organic/hybrid semiconductor to the radiation emitted by the radioisotope Technetium-99;

FIG. 3C shows the chart of the current intensity variation in response of an organic/hybrid semiconductor to the radiation emitted by the radioisotope Lutetium-177;

FIG. 4 shows a first embodiment of a detection system according to the present invention;

FIG. 5 shows a further embodiment of a detection unit according to the present invention;

FIG. 6 shows another embodiment of a detection unit according to the present invention;

FIG. 7 shows a second embodiment of a detection system according to the present invention;

FIG. 8 shows a detail of the detection system of FIG. 7; and

FIG. 9 shows a third embodiment of a detection system according to the present invention.

### DETAILED DESCRIPTION

In the various figures, similar parts will be indicated with the same reference numbers.

The detection system object of the present invention is a coating having a thickness that can vary indicatively from about 100 microns to a few millimeters, for surfaces that can act as a contaminameter and/or spectrometer, for the detection of any contaminants.

The detection device consists, as more fully defined below, of a flexible and scalable extended area sheet of ionizing radiation detectors.

The sheet can be used for the temporary covering of room surfaces, objects, tools, or devices in contact with unsealed radioactive sources, which can guarantee a more careful and visible monitoring of radioactivity in rooms subject to exposure. An insulating coating that can punctually and in real-time signal any contamination, which can be cleaned for immediate reuse and which therefore facilitates the decontamination phases of the affected area, and with production costs suitable for frequent replacement.

The detection system 1 is suitable for the detection and identification of potential surface contaminations, allowing punctual localization thanks to the integrated LEDs with which it is provided and/or to a screen display. The materials of which it is made allow it to be “washed” directly using common tools used for decontamination, allowing immediate reuse without the need for prompt replacement.

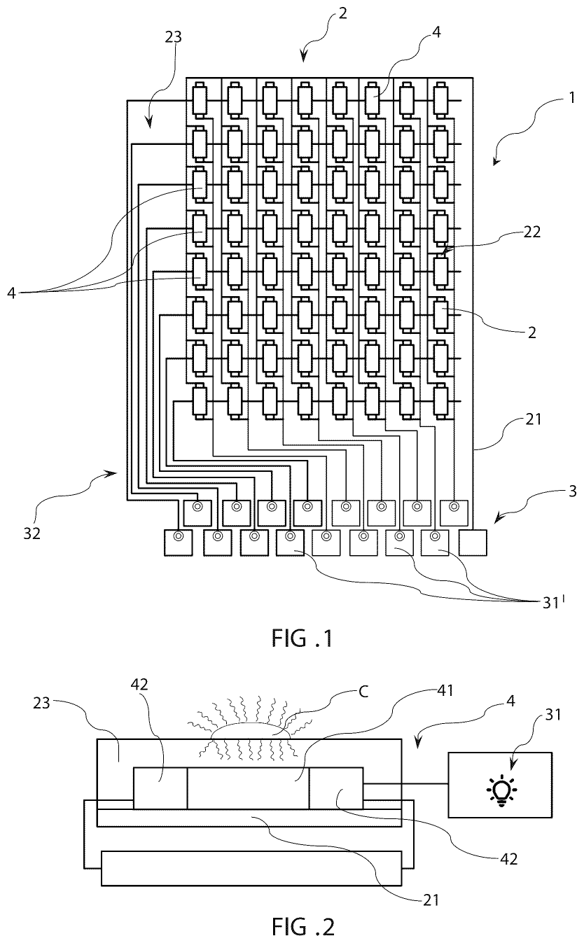

With reference to FIG. 1, a detection system 1 can be seen comprising a detection unit 2 and an interaction terminal 3, for displaying or communicating the readings made by the detection unit 2.

The detector unit typically consists of a multilayer detector sheet of ionizing radiation. In the exemplary schematic embodiment shown in FIG. 8, an 8×8 module is observed, on which a plurality of pixels are installed, with a mi llimeter dimension, which can be repeated and placed side by side until they cover a surface of 40 cm×40 cm and with a thickness between 0.5 and 5 mm. In modular terms, it is also possible to provide, in other embodiments, tiles having a size of 20×20 which can be placed side by side.

Each detection unit 2, as mentioned, can be made using low-cost liquid phase deposition techniques (blade-coating, drop casting, ink-jet printing, bar-coating, spray coating) or even from the vapor phase (thermal vacuum deposition, chemical vacuum deposition) of organic and hybrid semiconductors (based on perovskites) on a flexible substrate.

Each detection unit 2 consists of a support substrate 21, a detection layer 22, which can have a thickness in the range of 100 nm-20 μm, arranged on the support substrate 21, and an insulating film 23, generally made of impermeable insulating polymer, such as mylar, perylene, silicone polymers, arranged above said revealing layer 22, on which the contaminant agent C can be arranged.

The support substrate 21 is typically made of PET, PEN, or Kapton®. In any case, in other embodiments different and flexible substrates can be used, so that they can also be adaptable to irregular surfaces, such as, for example, various parts of the body in the case of applying the system as a personal dosimeter or a monitoring and tracking system of radiation, worn by the patient during diagnostic tests or radiotherapy sessions with radiopharmaceutical infusion or beam.

The detection layer 22 comprises a plurality of detection sensors 4, better described below, arranged according to different geometries, depending on the specific application for which they are made.

In the case under examination, the detection sensors 4 are arranged in a matrix and the detection layer has a rectangular shape, however, it is clear that other shapes and geometries can be provided according to the detection needs, as will be better described hereinafter.

With reference also to FIG. 2, the detection layer 22 is composed, as mentioned, of a plurality of detection sensors 4, which can, in fact, be photoconductors, photoresistors, diodes, or semiconductor transistors, arranged so as to cover an active surface, which each comprise the organic and/or perovskite hybrid semiconductor material 41, arranged on the support substrate 21, and the metallization of the electric contact 42, which forms the electrodes 42, connected to the semiconductor material 41.

The organic and/or perovskite hybrid semiconductor material 41 used to make the detection sensors 4 has a high sensitivity to ionizing radiation. The semiconductor material 41 can be made by various structural chemical formulas. In particular, some examples of organic molecules used are listed below:
4-hydroxycyanobenzene (4HCB);
1,8-naphthaleneimide (NTI);
1,5-dinitronaphthalene (DNN);
6,13-bis(triisopropylsilylethynyl) pentacene (TIPS-pentacene);
bis(triisopropylgermylethynyl) pentacene (TIPGe-pentacene);
2,8-difluoro-5,11-bis (triethylsilylethynyl) anthradithiophene (diF-TES-ADT);
5,11-bis(triethylgermylethynyl) anthradithiophene (diF-TEG-ADT);
polymer poly(9,9-dioctyfluorene) blended with Bismuth(III) oxide (Bi2O₃) nanoparticles (NPs);
Dinaphtho[2,3-b: 2′,3′-f]thieno[3,2-b]thiophene (DNTT) and n-type organic semiconductors.

But also 3D and 2D hybrid metal-alkaline perovskites of structure:
generic ABX₃, in which A=Methylammonium (MA=CH₃NH₃⁺), Formamidinium (FA=CH(NH2)₂⁺), Cs⁺; B=Pb²⁺, Sn²⁺; X=I³¹, Br³¹, Cl³¹ (Specific materials used: MAPbl₃, MAPbBr₃, mixed: CS_{0.05}BUT_{1-y}AND_{and}Pb(I₁₋ₓ Brx)₃);
“2D-layered” hybrid perovskites with generic formula (R-NH₃)₂PbX₄; R belongs to the alkyl or aryl group. Examples used: PEA₂PbBr₄ it is ALWAYS₂Pbl₄, with PEA=C₆H₅C₂H₄NH₃⁺;
Inorganic perovskites, for example CsPbX₃ (con X=Cl, Br o I).

In general, semiconductor materials 41 based on the given chemical formulas are also referred to as “small molecule organic semiconductors”: heterocyclic conjugated oligomers and conjugated ring two-dimensional linear molecular compounds. They can also have repeating units (monomers) but their molecular weight is generally less than 1500. This is particularly true for organic molecules (with reference to the first list, compared to perovskite ones. Perovskites are metal-organic hybrid molecules composed in particular from: Organic cation (e.g., MA) and a metal atom (e.g., Pb), and halogen anion (I, Br or Cl).

The detection device 4 is then covered by the insulating film 23.

Each detection device 4, by means of the electrodes 42, is connected to a respective LED 31 possibly via pads or electric pads 31′. As can always be seen in FIG. 2, when a contact contaminant C is not arranged on the insulating film 23 at the semiconductor material 41 of a device 4 of the detection layer 22, the radiations emitted hit said semiconductor material 41, generating a current passing through the material itself, following the creation of electron-hole pairs due to the ionizing action of the incident radiation. The signal thus generated is suitably amplified by an electronic module (not shown in the figure) to drive, in the schematic example shown in FIG. 2, the respective LED 31.

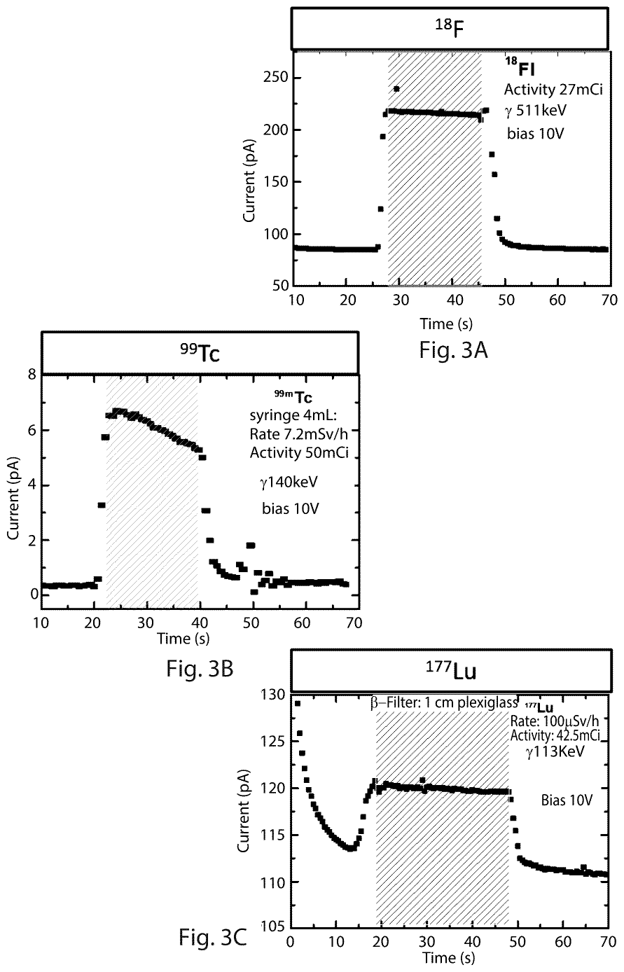

The semiconductor material 41 can be sensitive to contaminants of various types. In particular, with reference to FIGS. 3A, 3B, and 3C, the response to radiation of a contaminant C containing Fluorine-18, Technetium-99, and Lutetium-177, respectively, can be observed.

In particular, referring to FIG. 3A, it can be observed that the abscissa indicates the time, while the ordinate indicates the current in pico-Ampere (pA). As can be observed, the current passing through the semiconductor material 41 of a detection device 4 exposed to a radioactive contaminant C of gamma rays at an energy of 511 KeV passes from about 70 pA to 220 pA. This current is then amplified and used to drive a LED or a pixel of a screen (as better described below) and to detect not only the presence of a contaminant but also to identify the detection sensor 4, which is affected by the contamination as close to the contaminant C placed on the insulating film 23.

Among other things, according to the energy spectrum emitted by the contaminant, the detector 4 will have a different response, thus being able to drive the respective LED 31 or pixel, so that it emits, for example, a different color or sequence of colors in the visible spectrum. This allows, therefore, to identify and distinguish the type of contaminant unambiguously, obtaining a further spectrometer function of the radiation being analyzed.

An analogous interpretation can be made with reference to FIGS. 3B and 3C, in which the capacity of the semiconductor material 41 to retain the information, i.e. the time of reset to initial conditions once exposure to radiation is over.

The detection unit 2, as mentioned, is connected to the interaction terminal 3. In particular, in the embodiment at issue, the interaction terminal 3 comprises a plurality of LEDs 31, connected by means of conductive tracks 32 and possibly the respective ones to the array of detection sensors 4, which constitutes the detection layer 22 of the detection unit 2.

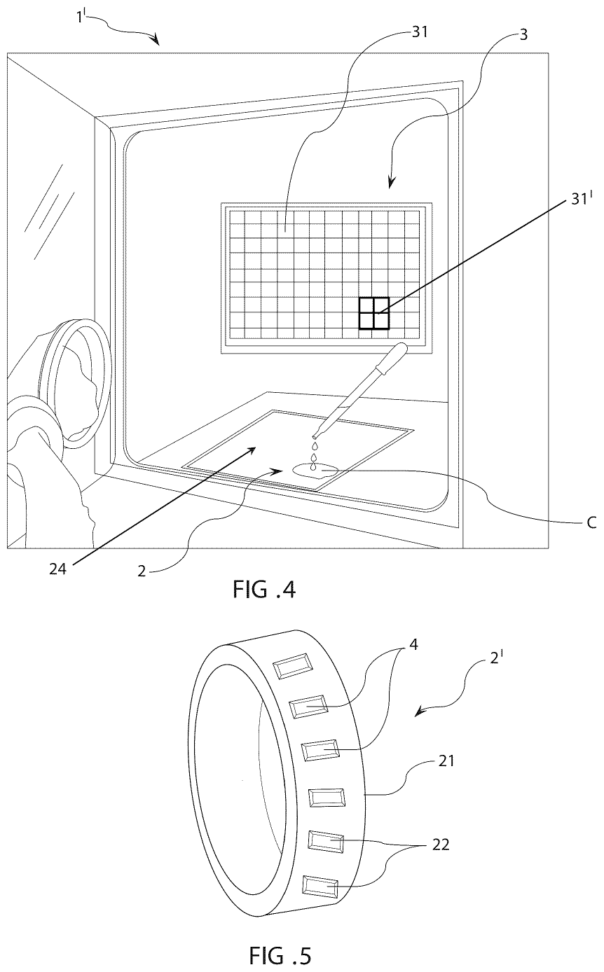

The general solution scheme described above can be applied in different ways and contexts. By way of example, reference is made to the set-up of FIG. 4, which shows a second embodiment of the detection system 1′, in which the detection unit 2 is a coating for surfaces in general. In this case, as can be seen, the detection unit 2 has a rectangular module, the surface 24 of which, as mentioned, is covered by the insulating film 23, is partially contaminated with a radioactive contaminant C, which could be, for example, a radiopharmaceutical, therefore containing radioactive isotopes.

The detection unit 2 is connected to the interaction terminal 3, which in the present case concerns a screen, which can consist of a matrix of LEDs 31 or pixels of a liquid crystal screen or other detail display technologies. The connection between the detection layer 22 and the interaction terminal 3 is by cable using suitably powered standard electronics.

The electric current generated, as mentioned above, in the detection sensors 4 affected by the proximity of the contaminant C charges an electric capacitance which, once a defined threshold has been exceeded, turns on the LED 31 corresponding to the contaminated pixel and/or transmits the information to the interaction terminal viewer 3.

Consider that the removal of the contamination (by cleaning the surface) brings the detector current to the reference levels, and the switching off of LED 31, and the reset of the display.

In this case, as can be seen, there is a spatial correspondence mapping of the surface 24 of the detection unit 2 with the interaction terminal 3, connecting the detection sensors 4 arranged in a matrix of the detection unit 2, with the LEDs 31 of the screen 33, grouped and connected so that the arrangement of the LEDs on the screen corresponds or maps the arrangement of the corresponding detection sensors 4 of the detection unit 2. In this way, as shown in the figure, the LEDs corresponding to the detection unit 4 affected by exposure to the radiation emitted by the contaminant C on the surface 24 of the detection unit 2 light up on the screen 33, allowing to supply not only the information of the fact that there has been a leak, which has been detected on the surface 24 of the detection unit 2, but also its position.

In a further embodiment, an alternative layout can be provided, which would provide for the “local” switching on of the LEDs 31, without the need for external cable connections to the screens. In this case, there is a first rigid and fixed support substrate (not shown in the figures) with the pixel LEDs 31, above which a second low-cost support substrate is arranged that is easily removable and/or replaceable, provided with the organic/hybrid sensors detectors 4 for the detection of traces of radioactivity.

In a further application or use variants of the present embodiment, the rectangular module constituting the detection unit 2 can be combined or connected to other similar modules, possibly side by side, so as to cover horizontal or even vertical surfaces of a potentially subject to contamination, so as to allow, similarly, not only the detection of contaminants, but also the location of the same. In particular, in other embodiments, instead of making a LED (or a pixel) of the screen correspond to a specific detection device 4 of the 2-module detection unit, it is possible to make each LED 31 correspond to an entire unit of 2-module detection, so as to have a much wider surface mapping.

As can be seen, the use of organic/hybrid technology detection sensors 4 which, deposited on a 21 millimeter support substrate and connected to integrated light sensors and/or LEDs, allow direct detection and real-time visual localization of the presence of radiation (generally made up of high energy photons, alpha and/or beta particles), allow more immediate and precise monitoring of any losses and/or presence of radioactivity in environments characterized by the diffusion of unsealed radioactive sources, allow the monitoring and centering of ionizing radiation beams. From an application point of view, in fact, the use of this detection unit considerably favors the monitoring of possible surface contamination and/or suspicious presence of radioactivity in circumstances that are not easy to inspect, such as in those places used for the disposal of waste subject to contamination, in hospital and/or airport environments, etc. This makes it possible to promote an improvement in the process of locating and removing deposited radioactivity, above all by obviating inconvenience in terms of handling of the devices currently used and proposing much lower production costs compared to those currently available in the market. It would also allow the centering of radiotherapy or diagnostic beams in real time, through wearable systems with a high degree of comfort for the patient thanks to the low weight and flexibility of the used materials.

As anticipated, today's systems for contamination measurement and dosimetry, and the respective detectors, generally include silicon diodes, proportional counters, G-M counters, point-detection scintillators, which however do not allow large area/surface detection.

In particular, in hospital environments (preparation hoods, warm rooms, rooms used for waste disposal) the perception and location of a possible contamination is almost completely left to the attention and operational experience of the personnel in charge. In this case, instead, the localization on the interaction terminal 3 is immediate and allows a certain intervention to remove the contaminating substance.

Furthermore, the detection unit 2 allows a real-time detection and makes the affected area visible through the LEDs 31 and the built-in sensors. Given the performance in terms of sensitivity at low doses, the coating with the insulating film 23 is easily decontaminable, making it possible to clean the contaminated area also with the absorbent materials commonly used for cleaning contaminated surfaces. The coating, i.e. the insulating film 23, can cover surfaces of various sizes according to the needs of use. The detection system 1 is therefore washable (or even disposable), readily reusable, with a life cycle of use ranging from a few days to months, and replaceable once it has become obsolete.

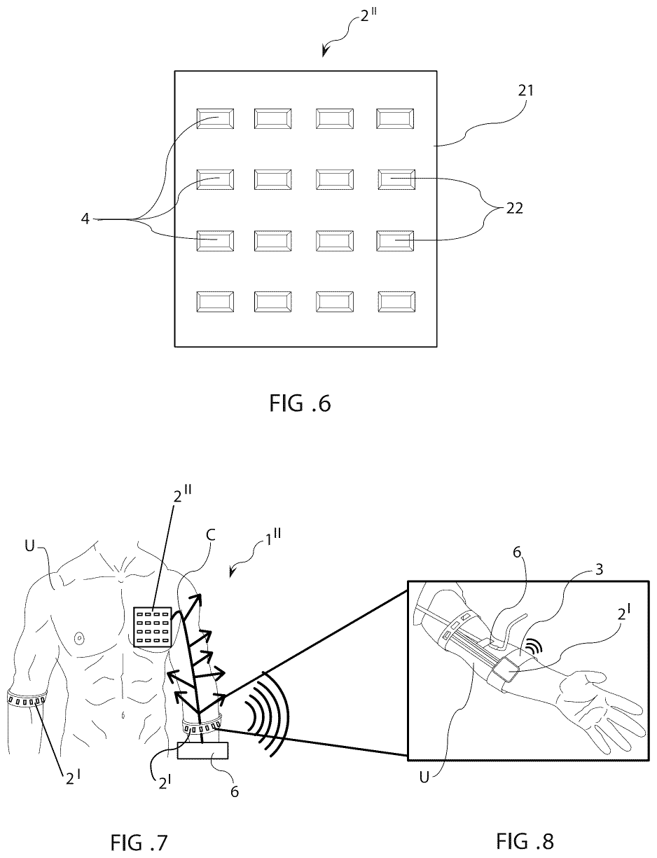

The structure and embodiment of the detection system according to the present invention can also be used in different configurations, such as the one shown in FIGS. 5 and 6, respectively representing the detection unit 2′ and 2″, respectively having the shape of a cuff and a plaster or patch. The bracelet-shaped detection unit 2′ and the patch-shaped detection unit 2″ are made to be worn by a user U, as shown in FIG. 7. In both cases, a support substrate 21 is always provided, which can have different shapes and geometries, as well as an active layer constituted by the detection sensors 4, which are also arranged according to different geometries.

In particular, with reference to FIG. 5, the detection unit 2′ can be seen as the support substrate 21 has the shape of a bracelet or band that can be worn by a user, while the detection sensors 4 are arranged around the external surface.

Instead, with reference to FIG. 6, the detection unit 2″ has a rectangular surface with a matrix arrangement of the detection sensors 4.

FIGS. 7 and 8 therefore show a third embodiment of the detection system 1″, particularly suitable for detecting the presence and diffusion of radiopharmaceuticals, i.e. drugs that can be inserted into the human body by infusion, as shown in particular in the FIG. 8 through a cannula 6.

By means of this embodiment, referring in particular to the diagram of FIG. 7, when the radiopharmaceutical is inoculated into the human body, for example through an intravenous infusion on the arm, its extravasation can be detected by means of the detection units 2′ and 2″. Therefore, it will be possible to reveal the presence of any extravasation of the radiopharmaceutical by means of the current difference read by the detection units 2′ or 2″.

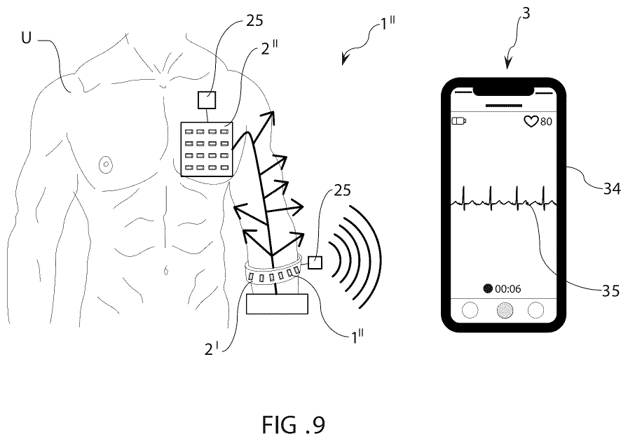

In the embodiment under examination, in particular, it can be observed that the user U wears the detection units 2′ or 2″, two on the arm and one on the chest. Still referring now to FIG. 9, a further embodiment of the detection system 1″ according to the present invention can be observed.

In this case, said detection system 1″ comprises two detection units and in particular a bracelet-shaped detection unit 2″, provided with transceiver means, indicated schematically with the reference number 25, capable of transmitting a relative signal to the readings of the detection sensors of the respective detection unit 2.

The detection system 1″ also comprises an interaction terminal 3, which in the embodiment shown is a mobile phone, which is capable of receiving the signals transmitted by the transceiver means of the detection unit 2, so as to show on the display 34 readings 35 of the different detection units 2′ and 2″.

As can be seen, this detection system 1″ is specifically designed to allow a sort of self-monitoring by a user U after the administration of an infusion of a radiopharmaceutical, for example in his own home environment, possibly also allowing memorization and/or transmission of how the same is spread within the body.

As can be observed, the detection system 1 has application in all those environments in which radioactive sources with a high risk of contamination and spill-out are handled, radiopharmaceuticals or unsealed radioactive powders but also radiation beams from accelerators or x-ray tubes.

The use of the detection system 1 improves the safety of the environments and consequently of the workers and users, making any contamination more perceptible in the places and on the most exposed surfaces (hot chamber, preparation hood), pre-empting the detection of the spill during the injection of radiopharmaceuticals and the monitoring of the incident dose on users undergoing beam radiotherapy sessions or X-ray examinations.

### Advantages

An advantage of the present invention is that of using innovative, highly performing, low-cost materials which can be processed on large surfaces, allowing the creation of sensitive coatings not otherwise achievable with traditional and consolidated electronic components on the market.

Another advantage of the detection unit according to the invention is that it allows the control of worktop contamination in, for example, the medical radiotherapeutic field, useful both for operators, who produce radiopharmaceuticals, and for hospitals, which administer oncological radiotherapy.

A further advantage is in the field of personal wearable dosimetry for patients undergoing radiotherapy and diagnostic tests with high comfort and low exposure to electrical risk thanks to the possibility of operating with low voltages, e.g. <10V. For example, the dosimeter can monitor the decay of a patient's dose even in the hours following administration, and at home, determining the real dose absorbed during the entire treatment.

Another advantage as a dosimeter is that it can be used to measure the dose locally in very irregular parts of the skin such as the nose or eyes, even with low-energy beams.

Another advantage of the present invention is that of allowing the control of the presence of radioactive material in areas of civil security (airports) or in radioactive landfills).

Finally, an advantage of the present invention is that of allowing low-cost (and therefore widely distributable) monitoring of the contamination of objects and foods in the event of radioactive fallout (e.g., Fukushima or Chernobyl).

The present invention has been described for illustrative but not limitative purposes, according to its preferred embodiments, but it is to be understood that modifications and/or changes can be introduced by those skilled in the art without departing from the relevant scope as defined in the enclosed claims.

1. Detection system of radiation emitted by a source (C) comprising:
at least one detection unit, having
a support substrate,
a detection layer,
arranged on said support substrate and comprising one or more detection sensors, to detect the radiation emitted by a contaminating agent (C) by generating an electrical signal following the interaction with the ionizing radiation, and
an insulating film, arranged on said detection layer, with which the contaminating agent (C) can come into contact, so that the ionizing radiation emitted by said agent is detected by one or more detection sensors; and
at least one interaction terminal, connected to said detection unit and configured to detect and communicate the position of said contaminant (C) based on the detection sensors affected by the ionizing radiation emitted by said contaminant (C).

2. Detection system according to claim 1, wherein each of said detection sensors comprises
a semiconductor material arranged on said support substrate, and
at least two electrodes, obtained by metallization, connected to the semiconductor material and to said interaction terminal.

3. Detection system according to claim 1, wherein
said detection sensors are configured to have a differentiated response according to the energy spectrum emitted by said contaminant (C) to drive said interaction terminal so as to distinguish the type of contaminant.

4. Detection system according to claim 1, wherein said semiconductor material is selected from one or more of the following:
4-hydroxycyanobenzene (4HCB);
1,8-naphthaleneimide (NTI);
1,5-dinitronaphthalene (DNN);
6,13-bis (triisopropylsilylethynyl) pentacene (TIPS-pentacene);
bis (triisopropylgermylethynyl) pentacene (TIPGe-pentacene);
2,8-difluoro-5,11-bis (triethylsilylethynyl) anthradithiophene (diF-TES-ADT);
5,11-bis (triethylgermylethynyl) anthradithiophene (diF-TEGADT);
polymer poly (9,9-dioctyfluorene) blended with Bismuth (III) oxide (Bi2O3) nanoparticles (NPs);
Dinaphtho [2,3-b: 2′, 3′- f] thieno [3,2-b] thiophene (DNTT) and organic n-type semiconductors.

5. Detection system according to claim 1, wherein said semiconductor material is of the perovskite hybrid type and is selected from one of the following:
having a generic ABX formulation3, wherein A=Methylammonium (MA=CH₃NH₃⁺), Formamidinium (FA=CH (NH₂)₂³⁰ ), Cs⁺; B=Pb²⁺, Sn²⁺; X=I⁻, Br⁻, Cl⁻, in particular MAPbI₃, MAPbBr₃, mixed: Cs_{0.05}FA_{1-y}MA_{y}Pb (I₁₋ₓ Brx)₃);
Hybrid perovskites having generic formula (R-NH₃)₂PbX₄, wherein R belongs to the alkyl or aryl group, such as PEA₂PbBr₄ and PEA₂PbI₄ , with PEA=C₆H₅C₂H₄NH₃⁺;
Inorganic perovskites, such as CsPbX₃ (with X=CI, Br or I).

6. Detection system according to claim 1, wherein
said support substrate is plastic, flexible, insulating and waterproof, and in that said insulating film is a waterproof insulating polymer, such as mylar, parylene, silicone polymers.

7. Detection system according to claim 1, wherein said detection unit is shaped as a rectangular or square module, intended to be arranged on a flat or vertical surface.

8. Detection system according to claim 1, wherein said detection unit has the shape of a bracelet or a band, so that it is wearable on a limb of a user (U), for the detection of radiation emitted by radiopharmaceuticals for the dose monitoring or the identification of radiopharmaceutical leaks.

9. Detection system according to claim 1, wherein said detection unit has the shape of plaster or patch, so that it can be applied on the surface of a user's body (U), for the detection of radiopharmaceutical leaks.

10. Detection system according to claim 1, wherein said detection unit has the shape of plaster or patch, so that it can be applied on the surface of a user's body (U), for the centering or the dosimetry of therapeutic and diagnostic beams.

11. Detection system according to claim 1, wherein said interaction terminal comprises a plurality of LEDs, forming a screen (33), configured for activating following the detection of radiation by one or more detection devices.

12. Detection system according to claim 11, wherein
each detection device is operatively connected to a respective LED,
wherein the arrangement of the LEDs on the screen corresponds to the arrangement of the corresponding detection sensors of the detection unit.

13. Detection system according to claim 11, wherein
it comprises a plurality of detection units each operatively connected to a respective LED,
wherein the arrangement of the LEDs on the screen corresponds to the arrangement of the corresponding detection units.

14. Detection system according to claim 1, wherein
said detection unit comprises transceiver means for transmitting signals detected by said detection sensors, and
in that said interaction terminal comprises a mobile phone, tablet and the like, equipped with a display, capable of receiving the signals transmitted by said transceiver means of the detection unit, so as to show on the display the readings of the detection units.

15. Detection unit for detecting a radioactive source (C), comprising
a support substrate,
a detection layer,
arranged on said support substrate, and
comprising one or more detection sensors, to detect the radiation emitted by a contaminating agent (C) by generating an electrical signal following exposure to the radiation, and
an insulating film, arranged on said detection layer, with which the contaminating agent (C) can come into contact, so that the ionizing radiation emitted by said agent is detected by one or more detection sensors.

16. Detection unit according to claim 15, wherein each of said detection sensors comprises
a semiconductor material arranged on said support substrate and
at least two electrodes, obtained by metallization, connected to the semiconductor material and to said interaction terminal.

17. Detection unit according to claim 1, wherein said semiconductor material is selected from one or more of the following:
4-hydroxycyanobenzene (4HCB);
1,8-naphthaleneimide (NTI);
1,5-dinitronaphthalene (DNN);
6,13-bis (triisopropylsilylethynyl) pentacene (TIPS-pentacene);
bis (triisopropylgermylethynyl) pentacene (TIPGe-pentacene);
2,8-difluoro-5,11-bis (triethylsilylethynyl) anthradithiophene (diF-TES-ADT);
5,11-bis (triethylgermylethynyl) anthradithiophene (diF-TEGADT);
polymer poly (9,9-dioctyfluorene) blended with Bismuth (III) oxide (Bi2O3) nanoparticles (NPs);
Dinaphtho [2,3-b: 2′, 3′- f] thieno [3,2-b] thiophene (DNTT) and organic n-type semiconductors.

18. Detection unit according to claim 15, wherein said semiconductor material is of the perovskite hybrid type and is selected from one of the following:
having a formulation generic ABX₃, wherein A=Methylammonium (MA=CH₂NH₃⁺), Formamidinium (FA=CH (NH₂)₂³⁰ ), Cs⁺; B=Pb²⁺, Sn²⁺; X=I⁻, Br⁻, Cl⁻, in particular MAPbI₃, MAPbBr₃, mixed: Cs_{0.05}FA_{1-y}MA_{y}Pb (I₁₋ₓ Brx)₃);
Hybrid perovskites having generic formula (R-NH₃)₂PbX₄, wherein R belongs to the alkyl or aryl group, such as PEA₂PbBr₄ and PEA₂PbI₄ , with PEA=C₆H₅C₂H₄NH₃⁺;
inorganic perovskites for example CsPbX₃ (with X=CI, Br or I).

19. Method for detecting radiations emitted from a source (C) comprising by means of a detection system according to claim furthercharacterized it comprises the following steps:
irradiating said insulating film with said source (C);
detecting the radiation of said radiation source (C) by means of one or more detection sensors of said detection layer;
detecting and communicating the position of said contaminating agent (C) by means of said interaction terminal, based on the detection sensors affected by the ionizing radiation emitted by said agent (C).
